(19) United States
(12) Patent Application Publication
(10) Pub. No.: US 2014/0294965 A1
(43) Pub. Date: Oct. 2, 2014

(54) COMPOSITIONS AND METHODS FOR EXFOLIATING PARTICLES

(71) Applicant: International Flora Technologies Ltd., Chandler, AZ (US)

(72) Inventors: James Steven Brown, Gilbert, AZ (US); Robert Kleiman, Sun Lakes, AZ (US); Jeffrey Addy, Phoenix, AZ (US)

(21) Appl. No.: 14/228,146

(22) Filed: Mar. 27, 2014

Related U.S. Application Data
(60) Provisional application No. 61/805,876, filed on Mar. 27, 2013.

(51) Int. Cl. A61K 8/02; A61Q 19/10; A61K 8/97; A61K 8/98

(52) U.S. Cl. CPC A61K 8/0241; A61K 8/987; A61Q 19/10; A61K 8/97
USPC 424/489; 424/539; 424/725

(57) ABSTRACT

Various embodiments provide compositions for exfoliating particles and methods for their manufacture. In one exemplary embodiment, the exfoliating particles may comprise a derivative of a botanically-sourced emollient, stearyl stearate, and at least one of: candelilla wax, is bran wax, sunflower wax, jojoba esters, carnauba wax, bees wax, corn wax, a saturated wax-ester, castor wax, ouricury wax, hydrogenated lanolin, and a hydrogenated triglyceride wax, Exemplary methods for producing the exfoliating particles may comprise melting, combining, and/or homogenizing the components of the exfoliating particles and cooling the resultant mixture in a process to form particles of a desired shape and/or size. The exfoliating particles may be for topical use and may be stable in personal care compositions.

### CROSS-REFERENCE TO RELATE!) APPLICATION

This application claims the benefit of U.S. Provisional Application Ser. No. 61/805,876, tiled Mar. 27, 2013, and incorporates the disclosure of such application in its entirety by reference. To the extent that the present disclosure conflicts with the referenced application, however, the present disclosure is to be given priority,

### BACKGROUND

The outermost layer of the epidermis consists of the stratum corneum and provides the barrier function of the skin. The stratum corneum is a complex biological structure comprising a matrix of cells, proteins such as keratin, and an intercellular lamellar lipid bilayer comprising free fatty acids and ceramides. Exfoliation of the stratum corneum, also called desquamation, occurs naturally in part through enzymatic processes wherein proteases cause proteolysis of intercellular contacts, resulting in the loss of external layers of cells such as corneocytes.

The renewal of the stratum corneum through ongoing exfoliation and regeneration from underlying layers of the epidermis contribute to healthy skin and the associated attractiveness of the skin. Exfoliating chemical or mechanical agents may increase the exfoliation of the stratum corneum and epidermal cell turnover an contribute to improved health of the skin. Cosmetic particles formulated to provide a mechanical exfoliating, effect may be problematic for a lack of stability, integrity of shape, surface precipitation, leakage of additives such as pigments and other disadvantageous features when incorporated into topical cosmetic formulations.

### BRIEF SUMMARY

Various embodiments provide compositions for exfoliating particles and methods for their manufacture. In one exemplary embodiment, the exfoliating particles may comprise a derivative, of a botanically-sourced emollient, stearyl stearate, and at least one of: candelilla wax, rice bran wax, sunflower wax, jojoba esters, carnauba wax, bees wax, corn wax, a saturated wax-ester, castor wax, ouricury wax, hydrogenated lanolin, and a hydrogenated triglyceride wax. Exemplary methods for producing, the exfoliating, particles may comprise melting, combining, and/or homogenizing the components of the exfoliating particles and cooling the resultant mixture in a process to form particles of a desired shape and/or size. The exfoliating particles may be for topical use and may be stable in personal care compositions.

### DETAILED DESCRIPTION OF EXEMPLARY EMBODIMENTS

The present invention may be described in terms of functional block components and various processing steps. Such functional blocks may be realized by any number of components configured to perform the specified functions and achieve the various results. For example, methods and systems according to various aspects of the present invention may be practiced in conjunction with any number of systems and methods for producing cosmetic, personal care, and/or pharmaceutical formulations, and the systems described are merely some exemplary applications for the invention. Various representative implementations of the present invention may be implemented into any personal care product for topical use on the skin of an animal.

The particular implementations shown and described are illustrative of the invention and its best mode and are not intended to otherwise limit the scope of the present invention in any way. For the sake of brevity, conventional manufacturing, connection, preparation, sterilization, and other functional aspects of the system may not be described in detail. Various aspects of the invention provide methods and systems for making and using exfoliating particles. A detailed description of various embodiments is provided as a specific enabling disclosure that may be generalized to any application of the disclosed systems and methods in accordance with the various described embodiments.

Exemplary embodiments of the present invention provide exfoliating particles that promote the removal of an outermost surface of the skin when applied to the skin of the human or animal. In various embodiments, the exfoliating particles may function as a mechanical exfoliant having to surface of adequate hardness and abrasiveness for promoting removal of the outmost surface of the skin. For example, the action of rubbing the exfoliating particles onto the surface of the skin may slough away loose and/or dead skin cells, oil, and/or debris, may unclog skin pores, and may uncover healthier looking skin. In some embodiments, the exfoliating particles may further comprise chemical exfoliants that may be released onto the surface of the skin through the action of rubbing the exfoliating particle onto the surface of the skin. Accordingly, the exfoliating particle may cause exfoliation through mechanical exfoliation, chemical exfoliation, or both.

In various embodiments of the present invention, the exfoliating particles may comprise a derivative of a botanically-sourced emollient, stearyl stearate wax, and at least one of; candelilla wax, rice bran wax, sunflower wax, jojoba esters, carnauba wax, bees wax, corn wax, a saturated wax-ester, castor wax, ouricury wax, hydrogenated lanolin, and a hydrogenated triglyceride wax. In some embodiments, the exfoliating particles of the present invention may exhibit enhanced stability when implemented into personal care products as compared to cosmetic microspheres composed of hydrogenated triacylglyceride oils and/or small chain wax esters. For example, in various embodiments, the exfoliating particles may exhibit an acceptable melting point and/or the ability to maintain particle shape, size, solidity, and/or pigment color. In some embodiments, the exfoliating particle may have a melting point of about 55° C. to about 65° C. In some embodiments, the exfoliating particle may not adversely affect physical properties of the personal care product such as viscosity, color, pH, surfactant qualities, conditioning qualities, and the like. Further, the exfoliating particles may resist degradation, formation of surface precipitants, and/or blooming when combined with the personal care products.

Exfoliating particles, in accordance with various exemplary embodiments, may comprise the derivative of a botanically-sourced emollient. The botanically-sourced emollient may comprise any plant-based material that may soften or smooth the skin and promote a reduction in roughness, dryness, cracking, and/or irritation of the skin. Smoothing of the skin may be effected by the penetration of the emollient into the surface of the skin, such as the stratum corneum and upper layers of the dermis, by rubbing the emollient on the skin.

In various embodiments of the present invention, the botanically-sourced emollient may compose botanical lipid materials. For example, in some embodiments, the botanical lipid materials of the botanically-sourced emollient may comprise fatty acids, esters of tatty acids, tatty alcohols, esters of fatty alcohols, esters of fatty alcohols with fatty acids, sugar alcohols, isopropyl esters, wax esters and/or combinations thereof extracted from the seed oil of the jojoba plant (Simmondsia chinensis). In one embodiment; the botanically-sourced emollient may comprise jojoba esters. In some embodiments, the exfoliating particles may comprise about 1 wt. % to about 3 wt. % of jojoba esters.

Any suitable derivative of the botanically-sourced emollient may be prepared from the botanical lipid materials extracted from the seed oil of the jojoba plant, such as, for example: refined jojoba oil, partially hydrogenated jojoba oil, and/or fully hydrogenated jojoba oil. In various exemplary embodiments, the exfoliating particles may comprise about 0.1 wt. % (weight percent) to about 20 wt. % hydrogenated jojoba oil. in some embodiments, derivatives of the botanically-sourced emollient may comprise hydrolyzed jojoba esters, an alkoxylated jojoba wax, an alkoxylated and at least partially hydrogenated jojoba wax, an alkoxylated product of jojoba oil interesterified with hydrogenated jojoba oil, an isopropyl jojobate, jojoba alcohol and/or the like.

The botanically-sourced emollient, in accordance with various exemplary embodiments, may be subjected to various chemical and/or physical refining processes prior to the preparation of the derivative. Exemplary refining processes may retain and/or concentrate a portion of the botanically-sourced emollient, such as jojoba esters, apart from other parts of the plant or seed, in some embodiments, the refining process may comprise any conventional chemical refining process such as degumming, neutralization of free fatty acids, decolorization, and/or deodorization of the botanically-sourced emollient. Degumming may comprise insolubilization of phosphatides with water in the presence of acid, and removal of the solid phosphatides by decantation or centrifugation. Neutralization of free fatty acids in the botanically-sourced emollient, such as the botanical lipid material, may comprise the addition of a base such as sodium hydroxide and the separation and removal of the resulting soaps formed, such as by centrifugation. Decolonization may comprise treatment of the botanically-sourced emollient with activated bleaching clays with heat, such as 100° C., under vacuum with filtration. Deodorization may comprise removal of compounds in the botanically-sourced emollient that may provide odor and/or flavor and may comprise the use of a conventional deodorizer apparatus that may use heat and steam to remove the odor and flavor compounds. The various refinement processes may be performed separately or in a continuous process and one or more refinement process may be used. In some embodiments, however, the botanically-sourced emollient may be used in a crude form without refinement.

Exfoliating particles, in accordance with various exemplary embodiments, may comprise stearyl stearate wax. Stearyl stearate wax is a fatty alcohol ester produced by the esterification of stearic acid and stearyl alcohol. The stearic acid may be extracted from the tricylglyceride oil produced by the palm plant Elaeis guineensis. The stearic acid may be subjected to a reduction reaction to produce stearyl alcohol. Stearyl stearate wax may have a melting point between about 55° C. to about 65° C. In various embodiments, the exfoliating particle may comprise about 0.1 wt. % to about 98 wt. % of stearyl stearate wax. In one embodiment, the exfoliating particle may comprise about 90 wt. % to about 95 wt. % of stearyl stearate wax.

The exfoliating particles, in accordance with various exemplary embodiments, may comprise any suitable wax obtained from a plant or animal including, but not limited to, candelilla wax, rice bran wax, sunflower wax, jojoba esters, carnauba wax, bees wax, corn wax, a saturated wax-ester, castor wax, ouricury wax, hydrogenated lanolin, and a hydrogenated triglyceride wax. In some embodiments, the saturated wax-ester may comprise behenyl behenate and/or stearyl behenate. In various embodiments, the waxes may be crude or further refined.

In an exemplary embodiment, the exfoliating particle may comprise candelilla wax obtained from the plant Euphorbia cerifera (candelilla shrub), wherein the candelilla wax may be extracted from the leaves of the candelilla shrub. In various embodiments, the exfoliating particle may comprise about 0.1 wt. % to about 50 wt. % candelilla wax. In one embodiment, the exfoliating particle may comprise about 4 wt. % to about 7 wt. % of candelilla wax. The candelilla wax may have a melting point above 68° C.

Exfoliating particles, in accordance with various exemplary embodiments, may further comprise a functional additive that may be entrapped, entrained. suspended, or otherwise disposed in or on the exfoliating particles. For example, the functional, additive may comprise, but not be limited to, one or more of alpha- and beta-hydroxy acids, amino acids, antibiotics, anti-fungals, antimicrobial agents, anti-perspirants, botanical extracts, colorants, cooling agents, cosmetically active ingredients, deodorants, depilatories, dermatologically active agents, detergents, dyes, emollients, enzymes, essential oils, flavors, fragrance fixatives, fragrances, fruit and/or vegetable extracts and/or juices, glitters, humectants, hyaluronic acid, mechanical exfoliants such as almond meal, peptide combinations, peptides, pharmaceutical preparations, pigments, preservatives, probiotics, provitamins, proteins, skin protectants, skin whiteners, soaps, styptics, sunless tanners, sunscreens, synthetic emollients, tocopherol, vitamins, and warming agents. The functional additive may be added, to the exfoliating particles in an effect amount. In some embodiments, the exfoliating particle may comprise about 0.1 wt % to about 4 wt. % of the pigments.

Methods for preparing the exfoliating particles, according to various aspects of the present invention, may comprise subjecting the botanically-sourced emollient to one or more refinement processes. The botanically-sourced emollient may be subjected to the refinement process prior to the formation of the derivative. In one embodiment, the botanically-sourced emollient may comprise jojoba esters that may be hydrogenated. according to known methods to produce hydrogenated jojoba esters. In some embodiments, the components of the exfoliating particles comprising the derivative of the botanically-sourced emollient, the stearyl stearate, and at least one of the: candelilla wax, rice bran wax, sunflower wax, jojoba esters, carnauba wax, bees wax, corn wax, the saturated wax-ester, castor wax, ouricury wax, hydrogenated lanolin, and a hydrogenated triglyceride wax may be heated to a temperature that meets or exceeds their melting temperatures. In some embodiments, each of the components of the exfoliating particles may be heated separately and then combined to form a liquefied mixture. In other embodiments, each of the components of the exfoliating particles may be combined in their solid form and then melted together by heating to form the liquefied mixture. In some embodiments, one or more functional additives may be added to the liquefied mixture.

The liquefied mixture may then be cooled and formed by known methods into solid exfoliating particles of a pre-selected shape such as beads, spheres, microspheres or the like of any desired size as described, for example, in U.S. Pat. No. 496,044, issued April 25, 1893 (now expired) and U.S. Pat. No. 2,714,224, issued Aug. 2, 1955 (now expired). However, the liquefied mixture may be formed into any desired shape including bars. In some embodiments, one or more functional additives may be added to the formed exfoliating particles, such as to the outside surface.

The exfoliating particles, in various aspects of the present invention, may be formulated to contain a pre-selected amount of each component by weight. For example, in some embodiments, the exfoliating particles may comprise about 0.1 wt. % to about 20 wt. % of fully hydrogenated jojoba esters. In some embodiments, the exfoliating particles may comprise about 0.1 wt. % to about 98 wt. % of stearyl stearate wax. For example, the exfoliating particle may comprise about 90 wt. % to about 95 wt. % of stearyl stearate wax. In some embodiments, the exfoliating particles may comprise about 0.1 wt. % to about 50 wt % of the at least one of the: candelilla wax, rice bran wax, sunflower wax, jojoba esters, carnauba wax, bees wax, corn wax, a saturated wax-ester, castor wax, ouricury wax, hydrogenated lanolin, and. the hydrogenated triglyceride wax. For example, the exfoliating particle may comprise about 4 wt. % to about 7 wt. % of candelilla wax.

In various embodiments, the exfoliating particles may comprise: about 1 wt. % to about 3 wt. % of a botanically-sourced emollient, about 90 wt. % to about 95 wt. % of stearyl stearate wax; and about 4 wt. % to about 7 wt. % of candelilla wax. In some embodiments, the botanically-sourced emollient may comprise jojoba esters. In this embodiment, the exfoliating particles may comprise about 1 wt. % to about 3 wt. % of the jojoba esters.

In an exemplary embodiment, the exfoliating particles may be incorporated into any suitable personal care product. In some embodiments, the exfoliating particles may be substantially suspended in the personal care product. In various embodiments, the personal care product may comprise body washes, facial cleaners, lotions, topical formulations, gels, creams, or any other suitable medium for delivering the exfoliating particles proximate to the surface of the skin. The exfoliating particles in accordance with exemplary embodiments of the present invention, may be incorporated into the personal care product according to the aesthetic and functional goals of that personal care product, in some embodiments, the exfoliating particles may maintain their hardness within the personal care product as substantially the same as the hardness of the exfoliating particle prior to its incorporation into the personal care product The ability of the exfoliating particles to maintain their hardness may sustain, and/or enhance their ability to exfoliate the skin in the presence of the personal care product.

In the foregoing description, the invention has been described with reference to specific exemplary embodiments. Various modifications and changes may be made, however, without departing from the scope of the present invention as set forth. The description and figures are to be regarded in an illustrative manner, rather than a restrictive one and ail such modifications are intended to be included within the scope of the present invention. Accordingly, the scope of the invention should be determined by the generic embodiments described and their legal equivalents rather than by merely the specific examples described above, for example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present invention and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition. system, or apparatus that comprises a list of elements does not include only those elements recited, but may also include other elements not expressly listed or inherent to such process, method, article, composition, system, or apparatus. Other combinations and/or modifications of the above-described structures, arrangements, applications, proportions, elements, materials or components used in the practice of the present invention, in addition to those not specifically recited, may be varied or otherwise particularly adapted, to specific environments, manufacturing specifications, design parameters or other operating requirements without departing from the general, principles of the same.

The present invention has been described above with reference to an exemplary embodiment. However, changes and modifications may be made to the exemplary embodiment without departing from the scope of the present invention. These and other changes or modifications are intended to be included within the scope of the present invention.

What is claimed is:

1. An exfoliating particle for topical application to the skin of a human or an animal, said particle comprising:
a derivative of a botanically-sourced emollient;
stearyl stearate wax; and
at least one of; candelilla wax, rice bran wax, sunflower wax, jojoba esters, carnauba wax, bees wax, corn wax, a saturated wax-ester, castor wax, ouricury wax, hydrogenated lanolin, and a hydrogenated triglyceride wax.

2. The exfoliating particle of claim 1, wherein the exfoliating particle is capable of being formed into a pre-selected shape.

3. The exfoliating particle of claim 2, wherein the pre-selected shape is a bead, sphere, or microsphere.

4. The exfoliating particle of claim 1, wherein the saturated wax-ester comprises at least one of behenyl behenate and stearyl behenate.

5. The exfoliating particle of claim 1, wherein the derivative of the botanically-sourced emollient is an at least partially hydrogenated jojoba ester.

6. The exfoliating particle of claim 5, wherein the derivative of the botanically-sourced emollient is a fully hydrogenated jojoba ester.

7. The exfoliating particle of claim 6, wherein the fully hydrogenated jojoba ester comprises about 0.1 wt % to about 20 wt % of the exfoliating particle.

8. The exfoliating particle of claim 1, wherein said particle comprises about 0.1 wt. % to about 50 wt. % of the at least one of the: candelilla wax, rice bran wax, sunflower wax, jojoba esters, carnauba wax, bees wax, corn wax, a saturated wax-ester, castor wax, ouricury wax, hydrogenated lanolin, and the hydrogenated triglyceride wax.

9. The exfoliating particle of claim 8, wherein said particle comprises about 4 wt. % to about 7 wt. % of the candelilla wax.

10. The exfoliating particle of claim 1, wherein the candelilla wax comprises a crude extract from the plant Euphorbia cerifera.

11. The exfoliating particle of claim 1, wherein the candelilla wax comprises an extract from the plant Euphorbia cerifera that is further refined.

12. The exfoliating particle of claim 1, wherein the stearyl stearate wax comprises about 0.1 wt. % to about 98 wt. % stearyl stearate wax.

13. The exfoliating particle of claim 1, wherein, the exfoliating particle is an ingredient in a personal care product adapted for care of human or animal skin.

14. The exfoliating particle of claim 13, wherein the personal care product is at least one of a body wash, a facial cleanser, and a lotion.

15. The exfoliating particle of claim 13, wherein the exfoliating particle resists degradation when combined into the personal care product.

16. The exfoliating particle of claim 13, wherein the exfoliating particle resists the formation of surface precipitates when combined into the personal care product.

17. The exfoliating particle of claim 1, further comprising at least one functional additive.

18. The exfoliating particle of claim 17, wherein the functional additive is at least one of a pigment, a fragrance, a vitamin, a preservative, an antioxidant a moisturizer, and an emollient.

19. The exfoliating particle of claim 1, wherein the exfoliating particle promotes the removal of an outermost surface of the skin when applied to the skin of the human or animal.

20. An exfoliating particle for topical application to the skin of a human or an animal said particle comprising:
about 1 wt. % to about 3 wt. % of a botanically-sourced emollient,
about 90 wt. % to about 95 wt. % of stearyl stearate wax; and
about 4 wt. % to about 7 wt. % of candelilla wax.

21. The exfoliating particle of claim 20, wherein the botanically-sourced emollient comprises a jojoba ester.

22. The exfoliating particle of claim 20, further comprising a pigment,

23. The exfoliating particle of claim 22, wherein said particle comprises about 0.1 wt. % to about 4 wt. % of the pigment.

24. The exfoliating particle of claim 20, wherein the melting point of said particle is about 55° C. to about 65° C.

25. A method for producing an exfoliating particle for topical application to the skin of a human or an animal, said method comprising the steps of:
melting and combining at least one of a botanically-sourced emollient and a derivative of a botanically-sourced emollient, a stearyl stearate wax, and at least one of: candelilla wax, rice bran wax, sunflower wax, jojoba esters, carnauba wax, bees wax, corn wax, a saturated wax-ester, castor wax, ouricury wax, hydrogenated lanolin, and a hydrogenated triglyceride wax to produce a liquefied mixture; and
forming exfoliating particles upon cooling of the liquefied mixture.

26. The method of claim 25, wherein the derivative of the botanically-sourced emollient comprises an at least partially hydrogenated jojoba esters.

27. The method of claim 26, wherein the derivative of the botanically-sourced emollient comprises fully hydrogenated jojoba esters.

28. The method of claim 27, wherein the fully hydrogenated jojoba esters comprises about 0.1 wt. % to about 20 wt. % of the exfoliating particle.

29. The method of claim 25, wherein said particle comprises about 0.1 wt. % to about 50 wt. % of the at least one of the: candelilla wax, rice bran wax, sunflower wax, jojoba esters, carnauba wax, bees wax, corn wax, a saturated wax-ester, castor wax, ouricury wax, hydrogenated lanolin, and the hydrogenated triglyceride wax.

30. The method of claim 25, wherein the stearyl stearate wax comprises about 0.1 wt. % to about 98 wt. % of the exfoliating particle.

31. The method of claim 29, wherein the particle comprises about 4 wt. % to about 7 wt. % of the candelilla wax.

32. The method of claim 25, further comprising combining a functional additive with the derivative of the botanically-sourced emollient, the stearyl stearate wax, and the at least one of the: candelilla wax, rice bran wax, sunflower wax, jojoba esters, carnauba wax, bees wax, corn wax, a saturated wax-ester, castor wax, ouricury wax, hydrogenated lanolin, and the hydrogenated triglyceride wax.

33. The method of claim 31, wherein the functional additive is at least one of a pigment, a fragrance, a vitamin, a preservative, an antioxidant, a moisturizer, and an emollient.

34. The method of claim 25, further comprising combining the exfoliating particle with a personal care product.

35. The method of claim 33, wherein the personal care product comprises at least one of a body wash, a facial cleanser, and a lotion.

36. The method of claim 25, further comprising homogenizing the liquefied mixture before forming the exfoliating particle.

37. The method of claim 25, wherein forming the exfoliating particle comprises forming microspheres.

38. The method of claim 25, wherein the saturated wax-ester comprises at least one of behenyl behenate and stearyl behenate.
